(19) United States
(12) Patent Application Publication
(10) Pub. No.: US 2022/0007970 A1
(43) Pub. Date: Jan. 13, 2022

(54) SYSTEM AND METHOD FOR PREVENTING AND PREDICTING THE RISK OF POSTURAL DROP

(71) Applicant: TECHBALANCE - SOLUÇÃO DIGITAL PARA REABILITÇÃO LTDA, Sao Paulo (BR)

(72) Inventor: Fabiana Mendes De ALMEIDA, Sao Paulo (BR)

(21) Appl. No.: 17/281,847

(22) PCT Filed: Oct. 2, 2019
(86) PCT No.: PCT/BR2019/050428

§ 371 Date: Mar. 31, 2021

(30) Foreign Application Priority Data
Oct. 5, 2018 (BR) 10 2018 070596 2

(51) Int. Cl. A61B 5/11; A61B 5/00; G16H 10/20; G16H 50/30

(52) U.S. Cl. CPC A61B 5/1117; A61B 5/112; A61B 5/6801; A61B 2562/0219; G16H 10/20; G16H 50/30; A61B 5/0002

(57) ABSTRACT

The present invention patent application for a system and method for preventing and predicting the risk of postural drop is in particular aimed at the health innovation sector, hardware engineering, software engineering, medical engineering, biotechnology, and more specifically to the field of wearable electronic systems for preventive medicine. The inventive solution is in the composition of solutions involving method and system enhanced through the use of specific signal processing, providing the clinical environment (4) and daily environment (5) with a low-cost, easy-to-understand solution that can be measured using graphs and numbers, as opposed to the existing clinical approach based on subjective evaluation, or the existing prior art that involves preventing drops exclusively from a motor perspective, and not systemically as recommended by scientific evidence.

### FIELD OF THE INVENTION

Deals with the present patent application for a system and method designed to prevent and predict the risk of postural drop, particularly aimed at the sector of health innovation, hardware engineering, software engineering, medical engineering, biotechnology and, more specifically, in the field of wearable electronic systems for preventive medicine.

### BACKGROUND OF THE INVENTION

Falls represent approximately 60% of all visits to the emergency department among the elderly and more than 50% of injury-related deaths annually (Haddad Y K, et at. Reducing Fall Risk in Older Adults. AJN The American Journal of Nursing 2018, 118(7):21-22. doi: 10.1097/01.NAJ0000541429.36218.2d) (accessed on 2Aug. 2018). Each year, an estimated 646,000 people die from falls globally, of which more than 80% are in low and middle-income countries (World Health Organization [WHO). Falls. 2016. Available from: http://www.who.int/mediacentre/factsheets/fs344/en/.).

Adults over 65 suffer the most fatal falls. Every year, 37.3 million falls require medical attention (World Health Organization [WHO). WHO global report on falls prevention in olderage. 2007 Available from: http://www.who.int/ageing/publications/Falls_prevention7March.pdf?ua=1). Despite more than ten years of research aimed at preventing falls among the elderly, the increase in the number of elderly people who died from falls exceeded 35% between 2005 and 2014 in the United States, and is expected to reach more than US$ 100 billion dollars in costs by 2030 and 100,000 fatal falls among the elderly annually (Centers for disease and control and prevention (CDC): Important Facts about Falls [Online]. Jan. 20, 2016. Available from URL: http://www.cdc.gov/homeandrecreationalsafety/falls/adultfalls. html Cited Jun. 15, 2016).

One third of the elderly population falls every year. Falls are responsible for 95% of hip fractures and a major cause of senile morbidity and mortality, with current costs estimated at more than $ 50 billion annually in the United States. For people aged 65 and over, the average cost of the health care system through injury through a fall is $ 14,056.00 (Centers for Disease Control and Prevention, National Center for Injury Prevention and Control, Division of Unintentional Injury Prevention. Important Facts about Falls. 2016, website: www.cdc.gov/steadi). Between the years 2015 and 2030, the number of elderly people over 60 was projected to increase by 56%, from 901 million to 1.4 billion, globally.

The population growth of the elderly most prone to falls, over 80 years old, is even more accelerated with projections that the population in this age group should reach 434 million individuals, that is, this population will triple by 2050 (World Health Organization [WHO). WHO global report on falls prevention in older age. 2007. Available from: http://www.who.int/ageing/publications/Fallsprevention7March.pdf?ua=1), making the socioeconomic impact of falls a worldwide public health concern. In Brazil, the expectation is that a fifth of the population will be made up of individuals over 65 years of age by 2060, with the growth rate of elderly people in Brazilian society expected to exceed 200% in the same period (Instituto Brasileiro de Geografia e Estatistica: Projeção da população do Brasil por sexo e idade para o periodo 2000-2060. Projeção da população das Unidades da Federa\tilde{c}ão por sexo e idade para o periodo de 2000-2030. ftp://ftp.ibge.gov.br/Projecao_da_Populacao/Projecao_da_Populacao_2013/nota_meto dologica_20 (accessed on 6Jul. 2018).

It is known that 20 to 30% of falls cause serious injuries and account for 10-15% of all visits to the emergency departments, being responsible for more than half of the hospitalizations caused by injuries among people over 65 years of age (World Health Organization [WHO]. WHO global report on falls prevention in older age. 2007. Available from: http://www.who.int/geing/publications/Falls_prevention7March.pdf?ua=1) . The main causes of hospitalization related to falls among the elderly are hip fractures, head injuries and injuries to the upper limbs. The long duration of hospitalization is considered the greatest cost for the health system. The number of days of hospitalization for falls is much higher than in other injuries, reaching 15 days and exceeding 20 days in cases of hip fractures (The University of York. The economic cost of hip fracture in the UK 2000. Health Promotion, England.), and the average number of days that the fall in the hospital adds to the elderly's hospitalization is 6.3 days (Joint Commission Preventing falls and fall-related injuries in health care facilities. Sentinel Event Alert. 2015; 28(55):1-5. [PubMed]). The length of hospital stay increases with increasing age and level of fragility and, in some cases, the elderly person can remain in the hospital until they die (The University of York. The economic cost of hip fracture in the UK 2000. Health Promotion, England.).

The post-fall consequences are also alarming, 20% of the elderly die within a year after a hip fracture. In addition, falls can also result in dependence and loss of autonomy, confusion, immobilization and depression, further increasing the motor risk for relapse of the fall, causing critical indirect costs for families with caregivers, treatments and recurring medical visits (World Health Organization [WHO]. WHO global report on falls prevention in older age. 2007. Available from: http://www.who.int/ageing/publications/Falls_prevention7March.pdf?ua =).

Rates of clinically treated falls are increasing and the elderly population is growing rapidly, which will result in a significant burden on the health system, with initial repercussions in Brazil demonstrated in the increase in the costs of health plans, despite the decrease in the lives served (Carneiro L A, Campino A C, Leite F, Rodrigues C G, Santos G M, Silva A R. Envelhecimento populacional e os desafios para o Sistema de sáude brasileiro [Internet]. São Paulo: Institute of Supplementary Health Studies; 2013. [cited 2015 May 1]. Available at: www.iess.org.br/envelhecimentopop2013.pdf).

In the case of Brazil, the scenario is even more worrying, as the country has one of the highest growth rates for the elderly in the world predicted for the next 40 years. In view of the weakness of the Public Health System (SUS) and the increase in the costs of private plans in the order of 18% for adherence, registered this year, the population may be without care and the health system may collapse, if appropriate prevention measures and resource management are not prioritized (Carneiro L A, Campino A C, Leite F, Rodrigues C G, Santos G M, Silva A R. Envelhecimento populacional e os desafios para 0 Sistema de saude brasileiro [Internet). São Paulo: Institute of Supplementary Health Studies; 2013. [cited 2015 May 1]. Available at: www.iess.orgbrlenvelhecimentopop2013.pdf).

Although accidents and costs caused by falls are common facts, they should not be seen as trivial or without consequence and there are still no adequate intervention and awareness solutions for the impact of the problem. The fact is that falls can be avoided, especially those related to aging and the clinical-hospital environment. The targeting of modifiable risk factors associated with evidence-based clinical interventions shows the best scientific results and includes participation in monitored physiotherapy sessions or an exercise program, improvement in postural tone, referral to specialists in vision, podiatry and review and management of medicines (Haddad Y K, et al. Reducing Fall Risk in Older Adults. AJN The American Journal of Nursing. 2018, 118(7):21-22. doi: 10.1097/01.NAJ.0000541429.36218.2d (accessed on 2Aug. 2018)). In this regard, the method proposed here focused on motor and clinical screening for the risk of falling and on promoting objective measures of the user's motor behavior, available in real time to support clinical decision-making, associated with continuous remote monitoring available to individuals under risk, with support for motor learning by vibration or beep of the wearable apparatus, suggesting motor correction whenever the gait trajectory is measured as dispersive, or under risk, promotes a systemic and interdisciplinary approach to preventive physical rehabilitation against falls.

### Status of Technique

The status of technique anticipates some previous patent applications. The solutions that basically determine the fall incident as an emergency are known to the public, especially based on the fall alarm, in the form of a message, a rescue button, among others. These techniques are usually based on inertial sensing from the analysis of movement in the three dimensions, comparing the acceleration and rotation of the movement with measures previously determined as risk. A recurring problem in these cases is the accidental alarm of the system, capturing sudden acceleration unrelated to the fall or the accidental fall of the device not attached to the user's body.

The document US 20110201972 A1 determines the false alarm in the occurrence of a fall prevention system that monitors the user's center of body mass in relation to a lower part of their body. Thus, depending on the minimum use of two sensors on the body, one on the center of body mass and one on the leg, the method estimates the risk of falling due to the threshold distance found between the area of the center of mass path and the polygon of feet support. This technique is limited to generating an estimate and depends on the operation of two or more sensors and extensive mathematical processing, without instantaneous or understandable results for the daily clinical approach and may not impact the individual at risk, if they do not understand or notice a change of attitude.

The most recent document, CN 103377541 A, suggests mathematical detection of the fall in a manner similar to that described above, but proposes real-time alert protection, before the 1 or 2 seconds in which the fall actually occurs, seeking to prevent fall or the effect of it, through remote alarm, vibration of anti-fall shoes and impact airbags. This solution depends, necessarily, that the elderly person is wearing or using all the devices for the preventive alarm. The solution is limited to seeking to change the user's attitude at the time of the fall or to minimize the clinical damage of the accident, if it is inevitable, despite the early alarm. This type of wearable solution also usually depends on an environment with adequate internet coverage, as the devices are integrated by wireless connection and possibly do not serve a population that grows on a large scale, especially low-income, limited access by cost or lack of internet coverage infrastructure. Another understanding is that this type of solution does not aim to improve the quality of life by preventing it, but rather to avoid the consequence of the imminent risk of falling.

Another known problem for position and displacement data using inertial sensing by low-cost electromechanical micro sensors, based on motion vectors, is the progressive and cumulative errors that compromise the accuracy of the speed and mainly position data, due to the integration of random gyroscopic noise and double integration and acceleration in accelerometers. Many solutions involving such inertial plants promote additional noise resistance with the use of filters, to transform the acceleration signal, first into speed and then into a displacement signal, removing the value of gravity z in the triortogonal axis of reference vectors. This solution has already demonstrated in scientific studies that it does not provide the necessary precision, maintaining part of the cumulative error, mainly in the acquisition of data for a long period of time (Sampaio, S., Massatoshi Furukawa, C., Maruyama, N.: Sensor fusion with low grade inertial sensors and odometer to estimate geodetic coordinates in environments without GPS signal. IEEE Lat. Am. Trans. (IEEE Latin America Magazine) 11(4), 1015-1021 (2013). doi: 10.1109/TLA.2013.6601744), requiring periodic system resets to minimize errors.

Another problem faced in the use of these wireless systems for characterization and human motor monitoring with 9 degrees of freedom, is the precision and remote access, in real time, of the displacement data. Here, the initial alignment of the Cartesian coordinates of reference to the georeferencing of the SNI is proposed, rotating to the NED oriented by the gravitational center of the Earth, using the accelerometers of the inertial center (9D cell sensors or Micro SiNS MEMS-Strapdown Inertial Navigation Systems on Microelectromechanical Systems) for leveling until only the z-axis accelerometers show a reading equal to the gravitational acceleration (-g) and the gyroscopes point to the geographic north (Sampaio, S., Massatoshi Furukawa, c., Maruyama, N.: Sensor fusion with low grade inertial sensors and odometer to estimate geodetic coordinates in environments without GPS signal. IEEE Lat. Am. Trans. (IEEE Latin America Magazine) 11(4), 1015-1021 (2013). doi: 10.1109/TLA.2013.6601744). Although this knowledge is public, it ensures the best accuracy of the position data by adequate filtering and referencing on the Cartesian axis in non-static initial alignment (Gao, W; Zhang, Y.; Wang, J. G. Research on initial alignment and self-calibration of rotary strapdown inertial navigation systems. Sensors. 2015;(15):3154-3171), enables the shortest delay of signal repercussion and the best real time reproduction for biofeedback, enabling an adequate clinical application (Bonora G, Catpinella I, Cattaneo D, Chiari L, Ferrarin M. A new instrumented method for the evaluation of gait initiation and step climbing based on inertial sensors: a pilot application in Parkinson's disease. J Neuroeng Rehabil. 2015;12(1) 45), (Schooten K S, Pijnappels M , Rispens S M, Elders P J M , Lips P, Daffertshofer A, et al. Daily-Life Gait Quality as Predictor of Falls in Older People: A 1-Year Prospective Cohort Study. 2016; 11(7). https://doi.org/10.1371/joumal.pone.0158623), especially for better accuracy of clinical measures and as part of improving the solution described in document BR 1020170169316.

Regarding the prediction of fall risk, there is an extensive scientific discussion with many positive insights related to the use of inertial sensors, but without defining the technique or product for the solution. In general, the status of technique calls for fall risk prediction, which in reality is the prediction of motor fall, because the risk of falling in the clinical context has a greater depth than the motor range, involves concepts beyond the system motor, but also visual and clinical risk measures already validated such as history of previous fall, use of risk medication, presence of visual diseases, among others (Ambrose A F, Paul G, Hausdorff J M Risk factors for falls among older adults: a review of the literature. Maturitas. 2013; 75:51-61).

Document US 20110152727 A1 also describes a fall prevention system for a user, comprising one or more sensors for connection to the respective body portions, each sensor being adapted to measure the movement of the respective body portion and translate the movement in a signal; and a controller adapted to receive the signal or signals and to determine the risk of falling, estimating a trajectory of the user's center of mass in relation to a trajectory of a lower part of the body.

The document U.S. Pat. no. 8,529,448 B2 proposes to warn the fall 2 or 3 hours in advance, by automatic identification of people who are at risk of falls through the use of a noninvasive, portable and inexpensive electronic device, and sensors equipped with software signal processing and predictive statistical algorithms that calculate stability and the presence of instability by difference between movement dispersion and a previously established dispersion index as a threshold. The two methods described above are limited to relate the motor information, not paying attention to the interdisciplinarity necessary for the prevention of falls, dispensing with the systemic clinical information, based on evidence (Bizovska L, Svoboda Z, Janura M, Bisi M C, Vuilletme N. Local dynamic stability during gait for predicting falls in elderly people: A one-year prospective study. PLoS ONE. 2018; 13(5): e0197091. https://doi.org/10.1371/journal.pone.0197091), important for the adequate assessment of the risk of falling.

For more than 10 years, in scientific publications on fall prevention without a decisive solution, due to the necessary complexity, interdisciplinarity and scalability (Centers for Disease Control and Prevention, National Center for Injury Prevention and Control, Division of Unintentional Injury Prevention. Important Facts about Falls. 2016, website: www.cdc.gov/stead), which should extend on several fronts of clinical intervention aimed at the prevention, treatment and monitoring of individuals at risk (Haddad Y K, et al. Reducing Fall Risk in Older Adults. AJN The American Journal of Nursing. 2018, 118(7):21-22. doi: 10.1097/01.NAJ0000541429.36218.2d (accessed on 2 Aug. 2018), (Ambrose A F, Paul G, Hausdorff J M Risk factors for falls among older adults: a review of the literature. Maturitas. 2013; 75:51-61). A recent cost-benefit study of three large-scale scientific interventions for fall prevention based on questionnaires and physical activity in the community evaluated the return on investment (RO!) of the implemented procedures, and found in the preventive physical intervention the highest ROI among the tested solutions, estimated at 509% (Carande-Kulis V, Stevens J A, Florence C S, Beattie B L, Arias I A cost-benefit analysis of three older adult fall prevention interventions. J Safety Res. 2015;52:65-70. pmid: 25662884).

Two other important clinical aspects related to falls are the presence of osteoporosis and step variability during the dual task, in situations that involve more than one mental activity such as walking, turning the head and talking. The low bone density and the presence of osteoporosis are related to hip fractures and there is a discussion about the possibility of bone fracture causing the fall. A recent clinical study with a large number of elderly people and research on risk of falling with motor information, bone density assessed by radiography and computed tomography, questionnaires and gait and balance measures indicates prevention involving exercises with slight peak acceleration, such as light running and strategies to improve gait in dual tasks, improve balance and muscle strength as the best solution for preventing falls, demonstrating that a method for preventing falls should not be limited to warning the fall in advance, but rather promoting the systemic physics practice (Johansson J The Healthy Aging Initiative: Prevention of Falls and Fractures. 2018. available at: https://www.researchgate.net/profile/Jonas_Johansson3/publication/324150029_T he_Healthy_Ageing_Initiative_Prevention_of_Falls_and_Fractures/links/5ac13e8aa6fd cccda65de851/The-Healthy-Ageing-Initiative-Prevention-of-Falls-and-Fractures.pdf).

In relation to stride variability, better evaluated balance and gait methods that will be integrated into this method proposed here for risk prediction and instant measures in the preventive rehabilitation clinic are: acceleration of the mean square root of the mid-lateral oscillation in the quiet position with eyes closed; 95% confidence interval of the total postural oscillation in the quiet position, number of steps, total time to complete the timed test of going; step time during walking, stride variability in the dual task and gait cadence (Johansson J The Healthy Aging Initiative: Prevention of Falls and Fractures. 2018. Available at: https://www.researchgate.net/profile/Jonas_Johansson3/publication/324150029_T he-Healthy-Ageing-Initiative-prevention-of-Falls-and-Fractures/links/5ac13e8aa6fdcccda65de851/The-HealthyAgeing-Initiative-Prevention-of-Falls-and-Fractures.pdf) (Montesinos L, Castaldo Rand Pecchia L. Wearable Inertial Sensors for Fall Risk Assessment and Prediction in Older Adults: A Systematic Review and Meta-Analysis. IEEE Transactions on Neural Systems and Rehabilitation Engineering. 2018; 26(3):573-582. doi: 10.1109/TNSRE.2017.2771383).

### The Invention

Fall prevention available in the system and method proposed here, is based on the interpretation of risk according to clinical data and available engines for screening fallers for the proper preventive clinical referral, support to preventive rehabilitation in the clinical environment and continuous monitoring of the user by wearable apparatus for use in daily activities. The system gauges and integrates clinical and motor data from an application that collects the patient's clinical history and posture and gait measurements on the cell through an inertial center over the center of body mass, head, torso or leg (cell or sensor). Thus, two main sources of data make up this method:

1) Clinical data instrumented in the application: questionnaire about the presence of a previous fall, use of risky motor medication, presence of urinary incontinence and vision problems, etc., and guidance for motor postures based on the MiniBestest clinical test, gold standard in clinical practice for screening fallers (Yingyong Yuddha A, Saengsiri Suwon A T, Panichaporrt W, et al. The Mini-Balance Evaluation Systems Test (Mini-BESTest) Demonstrates Higher Accuracy in Identifying Older Adult Participants With History of Falls Than Do the BESTest, Berg Balance Scale, or Timed Up and Go Test. J Geriatr Phys Ther. 2016;39(2):64-70.DOL 10.1519/JPT 00000000000000); -

2) Calculation of the risk of falling from an inertial sensor adapted in the body (cell or sensor) that performs the processing of the raw inertial signal and signal modeled by the Madgwick method (Madgwick S O, Harrison A J, Vaidyanathan R. Estimation of IMU and MARG orientation using a gradient descent algorithm. In Rehabilitation Robotics (ICORR), IEEE International Conference on (pp. 1-7), 2011), providing risk parameters based on gait, balance and Rosenstein's predictive method [Bizovska L , Svoboda Z, Janura M, Bisi Me, Vuilletme N Local dynamic stability during gait for predicting falls in elderly people: A one-year prospective study. P LoS ONE. 2018; 13(5): e0197091. https://doi.org/10.1371/journal.pone.0197091), (Thielo M Analise e classificacão de série temporais não estacionarias utilizando métodos não-lineares. Repositorio digital UFRGS 2000:13-98 http://hdl.handle.net/10183/12661 (accessed 2, Aug. 2018)) to support clinical decision-making.

### Problems Related to the State of the Technique Solved by the Invention

Important characteristics inherent to the status of the technique are improved in the present invention, presented in the following paragraphs.

1) Integration of clinical and motor information, through clinical analysis of risk history and assessment of gait and balance movement to prevent falls, to the detriment of techniques that assess only the motor condition.

2) Support for professional clinical decision in real time in the three areas of intervention with the patient: in the assessment (by screening the level of risk of falling and screening of fall-prone to fallers), in the treatment (objective measures of gait and balance, in graphs and numbers, available in the application for rehabilitation professionals to measure the patient's motor performance session by session), making it possible to generate a profile of the patient's motor behavior, to the detriment of current methods, involving subjective clinical tests and questionnaires, or objective measures by motor test devices restricted to the academic universe or high performance sports due to a high cost and high complexity in understanding the results, not accessible to the daily clinic and home environment.

3) Remote monitoring of the user's risk of the wearable device for continuous use, also available for the domestic environment, which allows the remotely monitoring of the user by the clinician and supports adaptive changes in the motor behavior of the user, through the vibration or beep of the apparatus in the event of risk, not being restricted to the attempt to prevent the fall, but rather, supporting the adaptive improvement of the user's motor system by beep or vibration, in the presence of the lowest risk measured by the system.

4) Initial alignment of the Cartesian coordinates of reference to the SIN rotating to the NED, allowing less error in the remote location of the user with a continuous wearable device.

5) Allows the user to monitor the movement of the wearable device continuously, remotely, both for the responsible professional and for a family member, who can follow the activities of that elderly person at home in real time and know if they have received a risk alarm in the form of beep or vibration of the apparatus.

6) Greater precision in position and displacement data, important for the accuracy of motor indexes and motor biofeedback technique by interaction with the signal, through signal modeling according to MARG quaternions, with data stabilization after 5 seconds, as observed in a pilot study (Almeida F M, Macedo F, Gil R C, Lima J J, Mochizuki L. Estabilometria Postural por Unidade de Medida Inercial (UMI). 7 Simposio de Analise de Sinais SIIMJSPS, 201 7. Available at: http://eventos.ufabc.edu.br/siimsps/files/id29.pdf (accessed on 6 Aug. 2018)), (Leite W, Couto H M, Liberal F J, Andrade M M. Avaliação cinematica comparative da marcha humana por meio de unidade inercial e Sistema de video. XXIV Congresso Brasileiro de Engenharia Biom\tilde{e}dica; 201413-17 Oct; Uberlãndia; p. 35.).

7) The method proposed here is not limited to capturing the emergency postural fall or to preventively warn of the possibility of a fall, but rather, it is a systemic method of care and prevention of fall that instruments the clinician and the patient in the fall risk screening, supports preventive rehabilitation treatment with objective gait and balance measures available to professionals in real time and continuously monitors the user's risk, also in the domestic environment, as support for continued treatment by vibration or beep of the apparatus wearable in the presence of dispersion of the walking and balance signal.

### Objectives of the Invention

The objectives of the invention are:

Screening of fallers with real-time results for adequate preventive clinical referral;

Objective instrumentation for clinical rehabilitation practice, for measurable management of motor performance and preventive fall rehabilitation, with results available in real time, in the clinical and domestic environment;

Sensitive alert by vibration or sound in a wearable device, seeking to change the motor attitude of the elderly, in their daily environment, as part of learning motor correction to prevent falls;

Remote monitoring of the user's risk using a wearable device, with results in real time, available for monitoring by clinicians and family members;

Assistance in the management of health resources by preventing falls and reducing costs with hospitalization generated by falls;

Improvement in the quality of life of the elderly, by preventing falls and encouraging supervised preventive physical activity.

### General Description of the Invention

This patent application refers to a new system and method for preventing postural fall by predicting risk, screening for individuals prone to fall and objective instrumentation of gait movement and postural balance in the clinical and domestic environment, providing instant results on the cell for clinicians and family members and/or alerting about the risk of falling directly to the user, by vibration or audible alarm in a wearable device. The method is based on the integration between motor data by inertial sensing, prediction of fall risk by dispersion analysis of the gait trajectory and screening of clinical risk in the application. The biotechnology and innovation sector notably needs this solution due to the great social and financial impact of falls, especially those related to aging and the hospital environment, in addition to the need for motor parameters objectives that assist in performance management and evolution of clinical rehabilitation practice.

### DESCRIPTION OF THE FIGURES

The invention will be explained below in a preferred embodiment, and for better understanding, references will be made to the attached drawings, in which they are demonstrated:

FIG. 1: Flowchart of the system showing the flow and composition of the data of the invention and how the results are shared with the clinician, the user and the family member;

FIG. 2: Flow diagram of the system showing how the data is processed and composed to generate the results, the processing autonomy in the offline and cloud environment and how the results are intended for the clinical environment for professional use and everyday environment for the user biofeedback and monitoring of family members;

FIG. 3: Flow of data modeling according to Madgwick's method of signal error modeling from quaternions in relation to the inertial navigation system (SNI);

FIG. 4: Schematic representation of the stability of human gait, according to the author. In this Figure, “the undisturbed dynamics of the xe (t) gait is represented by the red orbit with a radius ε. The ε limit represents a small distance to the disturbed gait dynamics x (t) for a small perturbation of size d (0) <^{δ}, which does not interfere with stability. (B) A schematic representation of ordinary stability (green line), asymptomatic stability (yellow line) and exponential stability (red line) within a section of time (blue dashed lines). Distance d (t) between the undisturbed xe (t) and the disturbed gait dynamics x (t) must be within radius c for the dynamics to be stable. In addition, the yellow arrow must approach the undisturbed path xe (t) (red central line) as t −>∞. In addition, the red arrow must approach the red exponential arrow, where d (t) ≤d (0) exp (λt) and the exponent of Lyapunov λ<0, when the dynamics are exponentially stable” (Bizovska L, Svoboda Z, Janura M, Bisi M C, Vuillerrne N Local dynamic stability during gait for predicting falls in elderly people: A one-year prospective study. PLoS ONE. 2018;13(5): and 0197091. https://doi.org/10.1371/joumal.pone.0197091). “The size of a disturbance is evaluated as the distance d (0)=II×(0)−xe (0) II between the disturbed point x (0) and the undisturbed point xe (0) in the reconstructed state space (see upper red vertical arrow in FIG. 1B of FIG. 4). The reaction to the disturbance is numerically defined as the temporal change, d (t)=II×(t)−xe (t) II, from the initial distance d (0) between the trajectory of the disturbed gait x (t) and the undisturbed trajectory of the gait xe (t)” (Bizovska L, Svoboda Z, Janura M, Bisi M C, Vuilletme N Local dynamic stability during gait for predicting falls in elderly people: A one-year prospective study. PLoS ONE. 2018;13(5): e0197091. https://doi.org/10.1371/joumal. pone. 0197091);

FIG. 5: Rosenstein fall prediction method, according to the calculation of the maximum Lyapunov exponent. The log of the expansion/contraction of the Euclidean distance between these points of the neighboring estimated trajectories of the march by max)′ 11, represents the rate of divergence or convergence of the march signal or the degree of local dynamic stability of the system;

FIG. 6: Fall risk level classification method involving clinical information data, motor information (objective gait and balance indexes) and Rosenstein risk prediction, in order to make a calculation of the risk framework and generate results involving the levels risk.

### DETAILED DESCRIPTION OF THE INVENTION

The “SYSTEM AND METHOD FOR PREVENTING AND PREDICTING THE RISK OF POSTURAL FALL”, object of this patent application, describes a system and method for preventing falls that has the purpose of offering an objective tool in the clinical and domestic environment, based on clinical and motor information for the assessment, screening and monitoring of the risk of falling and motor performance with encouragement to the practice of preventive physical rehabilitation available as an objective tool for health professionals and as a tool for remote control of risk, in the domestic environment, by family members and biofeedback to support motor correction for the user. The main objective of the method is to offer a systemic and interdisciplinary coverage to support clinical decision-making and remote user monitoring, to impact fall prevention, improve the quality of life of the elderly and intelligent management of health resources by decreasing the rate of hospitalizations and costs with falls in the hospital, clinical and domestic environment.

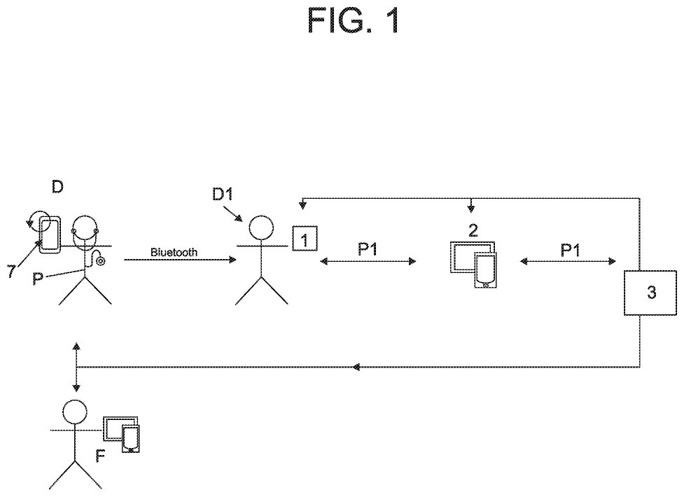

In FIG. 1, the system flowchart presents the patient (P) in the collection of DONE clinical data (D) and motors (D1) with the dedicated application and sensor (1) (cell or inertial sensor). In the application, the therapist (T) collects information processed by a binary method that classifies the prevention or absence of the risk assessed in a questionnaire about the previous history of risk of falls with positive or negative, integrating questions about previous fall, use of risk medication, urinary incontinence, among others. Still in the application, the therapist (T) asks the patient (P) to perform 14 tasks that include sitting and standing up, standing with eyes closed, under an uneven surface, walking, sitting and going back, among others (clinical test based on MiniBestest); used to evaluate anticipatory and compensatory reactions, sensory guidance and dynamic gait balance. During the performance of these tasks, the patient (P) uses the sensor (1) attached to the body, which automatically processes the inertial signal of the tasks to generate the risk indexes of falling, gait and balance, and classification of the level of risk at low, moderate or high risk, according to processing autonomy (PI) shown in the figure (offline (6) and cloud (3)), and part of the algorithms are embedded in the sensor (1) of the cell (2) and the another part is processed in the cloud (3), instantly in the presence of the wireless signal, via Bluetooth connection, or as soon as possible, as soon as the system enters the wireless coverage. This clinical solution extends to physical rehabilitation, as a measure of the patient's performance result in physical rehabilitation sessions and for home use, if the therapist (T) understands that the patient (P) remains at risk and needs to continually use the apparatus wearable in their daily activities. In this case, the patient (P) has their risk constantly controlled by continuous and automatic calculation of the risk, with partial processing autonomy (P1) between the apparatus and the cell (2) of the patient (P). For prevention, the Rosenstein index rate reduction trigger will trigger the vibration or beep of the apparatus to request the patient's motor attention (P). The user's motor behavior can also be monitored remotely, from cloud processing (3), by the therapist (T) or family (F).

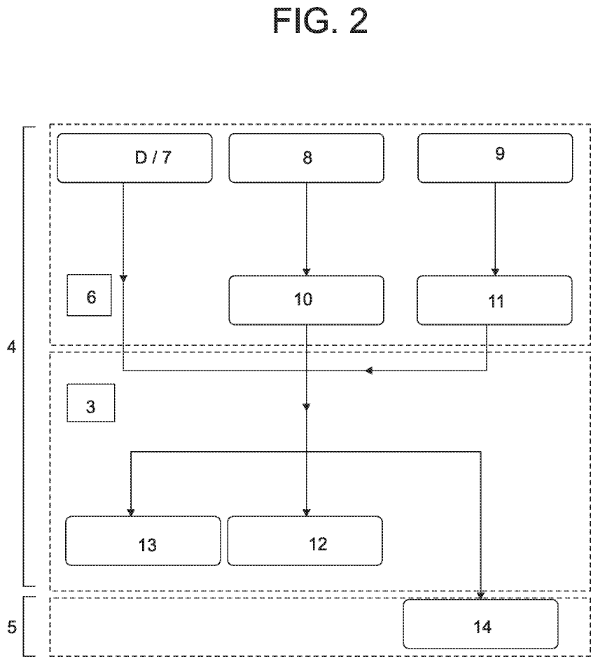

FIG. 2 shows a flow with a clinical environment (4) and a daily environment (5), with part of the clinical environment (4) being offline (6) and part in the cloud (3); among the related data are the clinical data (D) APP (7), the 9D inertial data (8), the quaternion fusion sensors (9), the 9D inertial data (8) communicating with the Rosenstein prediction of fall risk (10) and the quaternion fusion sensors (9) with the gait and balance algorithms (11), with a communication between these with the gait and balance indexes (12) available in real time to support preventive rehabilitation. In the clinical environment (4) there is also the screening (13) of fallers by risk level with the suggestion of clinical guidance, this communicator with the risk monitoring (14) in the domestic environment with preventive biofeedback by continuous use of a wearable device.

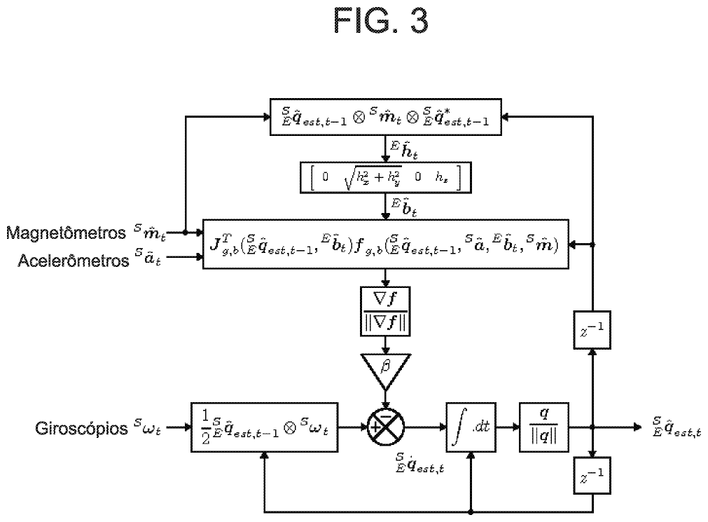

Thus, FIGS. 2 and 3 demonstrate how the results are achieved from the raw motion data from the 3 accelerometers (A1), 3 gyroscopes (G1) and 3 magnetometers (M1) of the inertial sensor (1) cell (2) or inertial sensor (1) coupled to the patient's body (P) in the clinical assessment of the risk of falling, or in the same way, continuously coupled to the domestic patient (P), according to two distinct flows:

1) Fusion of data from the 9 sensors of the MEMs plate of the cell (2) or sensor (1), by the Madgwick signal modeling method, by rotated quatemions (Yingyong Yuddha A, Saengsiri Suwon AT, Panichaporrt W, et al. The Mini-Balance Evaluation Systems Test (Mini-BESTest) Demonstrates Higher Accuracy in Identifying Leader Adult Participants With History of Falls Than Do the BESTest, Berg Balance Scale, or Timed Up and Go Test. J Geriatr Phys Ther. 2016;39(2):64-70. DOI: 10.1519/JPT 000000000). From the output of the modeled signal, there are the three motion vectors in the axes y (Pitch), x (Roll) and z (Yaw), agreed according to the initial leveling to the SIN rotated to the NED, forming the 3D-axes for data of angular movement, which provide information of attitude and orientation with better precision. The data is exported in real time by Bluetooth low energy for integration with the cell and application, which, in tum, has processing autonomy so as not to lose data in the absence of a wireless signal. At the same time, the inertial signal fused in the 9 degrees of freedom with autonomy for offline processing (6) exports the data for cloud processing (3) of the gait and balance calculations and Rosenstein for gait, balance and risk of falling. All information is integrated, clinical and motor, and made available for reading in real time in the application for the objective motor quantification of gait and balance indexes and classification of the level of risk of falling, with subsequent suggestion of adequate clinical guidance, biofeedback of vibration or beep, for the wearer of the device continuously wearable in the domestic and everyday environment, whenever their risk of falling, supervised by a continuous automatic risk assessment method, observes the dispersion of the signal from time to time by rendering the automatic algorithm, gait and balance suggesting risk. Given this information, the patient (P) can proactively correct their posture or seek specialized care. Family members (F) can also have access to the dedicated application to accompany their elderly family member, user of the wearable fall prevention device, to find out how long they spent sitting, lying down, if they did the requested walks or if they have received a risk alarm;

2) Calculation of prediction of risk of fall by the method of Rosenstein from the acceleration information of the three accelerometers (A1). In other words, disturbances in the dynamic gait system can be represented by calculating the local dynamic stability by reproducing the influence of these disturbances, in the estimated neighboring trajectories, which deviate from the original trajectory of the gait represented in the state space. Gait stability is maintained by the control of active and passive stiffness of the corrective neuromuscular system, through the strategy of recruiting active muscles combined with the motor adjustment of the joint torques necessary to maintain dynamic balance (Lockhart T E, Liu J 2008 Differentiating fall prone and healthy adults using local dynamic stability. Ergonomics 51, 1860-1872. doi: 10.1080/00140130802567079 (doi: 10.1080/00140130802567079″. “The “stable” gait can be pragmatically defined as one that does not lead to falls, despite disturbances” (Bruijn S M, Meijer O G, Beek P J, van Dieen J H (2013) Assessing the stability of human locomotion: a review of current measures. J R Soc Interface 10:20120999). The measure of local dynamic stability has already been demonstrated in a clinical study to be able to differentiate gait between elderly fallers, healthy elderly and young people (Lockhart TE, Liu J 2008 Differentiating fall prone and healthy adults using local dynamic stability. Ergonomics 51, 1860-1872. doi: 10.1080/00140130802567079, doi: 10.1080/00140130802567079). The capacity of the neuromuscular system to attenuate gait disturbances is the target of measuring local dynamic stability based on nonlinear dynamic theory.

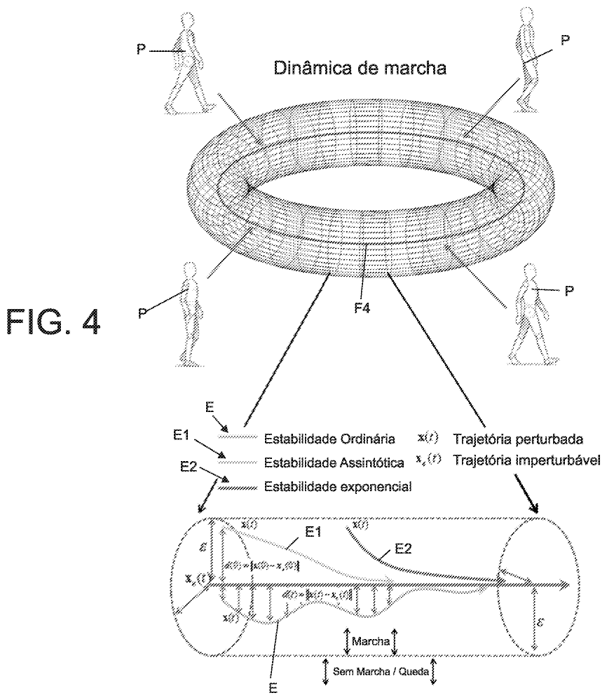

In FIG. 4 the author explains, through the schematic drawing, how gait stability is understood. That is, the flow (F4) shows the gait dynamics of a patient (P). Below, the graph (G2) illustrates the curves obtained for ordinary stability (E), asymptotic stability (E1)) and exponential stability (E2), where x(t) represents disturbed trajectory and x^{e}(t) represents imperturbable trajectory.

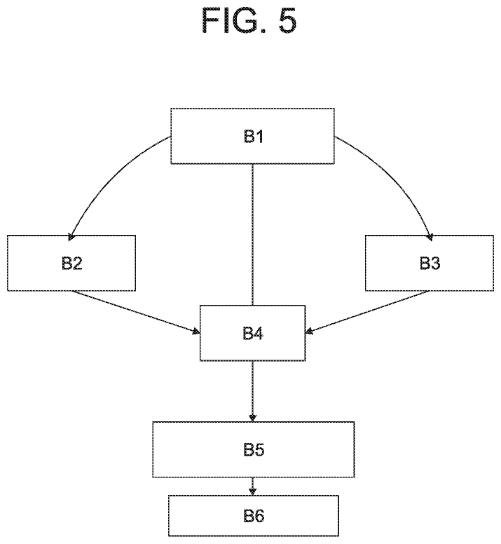

Lyapunov exponents of a trajectory measure the average rate of dispersion or conversion of nearby trajectories, in the context of human gait. If there is convergence of the signal, the stability of the system can be understood as good. However, the greater the divergence of the signal, the more chaotic the system is considered. Studies show that the Lyapunov Maximum Exponent measurement in short time series is ideal for calculating gait stability, to the detriment of samples over long time (World Health Organization [WHO). WHO global report on falls prevention in olderage. 2007. Available from: http://www.who.int/ageing/publications/Falls_prevention7March.pdf?ua=1). Rosenstein proposed an alternative approach to calculate the largest Lyapunov exponent (max λ1) from a short time series, with less noise influence. The Rosenstein method for calculating maxλ1 consists of the following steps, represented in the block flowchart of FIG. 5, where the block (B1) of original time series data (AP acceleration, 40 travel cycles) is represented; block (B2) (time delay—10 frames); block (B3) (incorporation dimension); block (B4) (reconstructed state space) submitted to the Rosenstein algorithm, which feeds the block (B5) (mean divergence between close trajectories) and from this to the walking stage (B6) (Lockhart TE, Liu J Differentiating fall-prone and healthy adults using local dynamic stability. Ergonomics. 2008; 51:1860-725): “State vectors are reconstructed from {xi} data. For each point i in the reconstructed space, the nearest neighbor j that is outside a time limit greater than the mean period of the series (estimated from the Fourier transform of the series), so that/i-j/ >average period. The temporal evolution of the distances between these points is followed up to a time limit Δt, accumulating the value of the logarithms of the distances and calculating the average of these values (dividing by Δt)” [19]. “The rocedure from (1) to (3) is repeated for different pairs of points, allocating the logarithm of the mean of these divergences as a function of the current value of Δt. The procedure is continued until Δt reaches a pre-established limit. Then a line is fitted to the resulting set of points—“( . . . ) the time versus the log of the Euclidean distance curve is calculated for all neighboring points of their respective trajectories” (Lockhart T E, Liu J Differentiating fall prone and healthy adults using localdynamic stability. Ergonomics. 2008;51:1860-72)—“( . . . ) the propensity of this line approaches the value of the greatest exponent of Lyapunov λ1 (Thielo M Análise e classifição de série temporais não estacionárias utilizando métodos não-lineares. Repositório digital UFRGS. 2000:13-98 http://hdl.handle.net/10183/12661 (accessed 2, Aug. 2018))” and “( . . . ) describes the rate at which the kinematic variability approaches the trajectory of the equilibrium movement or how much the slope curve of maxλ1 diverges from the average curve. The higher the maxλ1, the faster the divergence will increase and the worse the system's resistance to disturbances. Consequently, the higher maxλ1 indicates less local dynamic stability of the human motor control system” (Lockhart T E, Liu J Differentiating Jail prone and healthy adults using localdynamic stability. Ergonomics. 2008;51:1860-72) , that is, risk of falling”.

In recent clinical findings, time series dispersion measures representing gait cycle projection were considered to be potential for predicting the risk of falling when associated with clinical measures. The maximum Lyapunov exponent in short time series, computed from the linear acceleration of the torso in the medial-lateral direction, was associated with the decline in the rate of stability of falling individuals in a longitudinal study involving 131 elderly people. In this same study, the “pure” measure of)′1 was considered insufficient to predict the senile decline (Bizovska L, Svoboda Z, Janura M, Bisi Me, Vuilletme N Local dynamic stability during gait Jor predicting Jails in elderly people: A one-year prospective study. PLoS ONE. 2018;13 (5): and 0197091. https://doi.org/10.1371/joumal.pone.0197091), as proposed in document US 20110152727 A1, showing the relevance of this proposed method that includes the clinical variables measured in the application as validation for the physical-mathematical measures of risk, providing greater precision for these measures and promoting the interdisciplinary approach required for the prevention of falls.

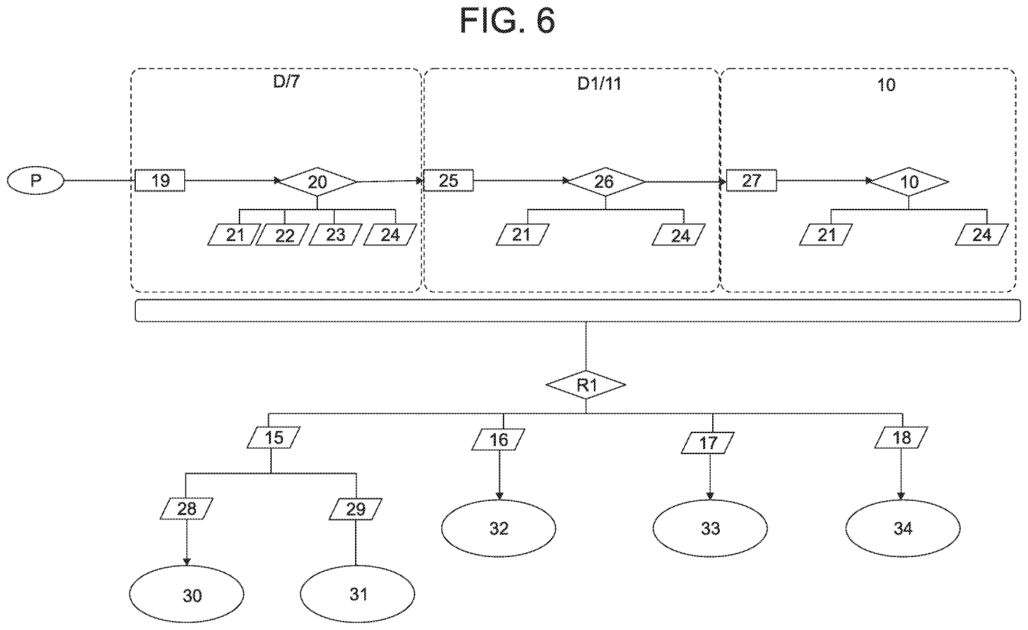

FIG. 6 shows the method proposed here based on the integration of clinical data (D) APP (7) (clinical information) and gait and balance algorithms (11) (objective gait and balance indexes), by application and processing of measured vectors of the inertial signal, used on the patient's body (P) for clinical testing—prediction of the level of risk of falling (10) (Rosenstein risk)—performed by a technician in the clinical environment (4) (rehabilitation professional) which refers the patient (P) to the most appropriate treatment. These combined items generate the calculation of the risk of falling, generating a result (R1) that can indicate high risk (15), moderate risk (16), low risk (17) or no risk (18).

In the clinical data (D) APP (7) table, the patient (P) is subjected to a test 1 (19) based on the clinical data (D) APP (7), generating the evaluation (20) and the results: risk of falling+=3 (21), risk of falling+=2 (22), risk of falling+=1 (23) and risk of falling+=0 (24); then, in the gait and balance algorithms (11), the patient (P) is subjected to a test 2 (25) based on these indicators, generating the “motor parameters” (26) and the results: risk of falling+=3 (21) and risk of falling+=0 (24); in the table prediction of the level of risk of falling (10) (Rosenstein) the patient (P) is submitted to a test 3 (27) that leads to the risk prediction (10B), with the results: risk of falling+=3 (23) and risk of falling+=0 (24).

After assessing and calculating the risk of falls, the result (R1) generated specifies in the table high risk (15) a value >6 where there are two options: high risk 12-9 (28) and high risk 8-6 (29). The high risk 12-9 (28) has the following procedures as an indication: “preventive physiotherapy+use of continuous wearable apparatus in the daily environment+medication review and eye exam” (30); the high risk 8-6 is indicated by the following procedures: “preventive physiotherapy+motor test in activities of daily living, for one week, at the end of rehabilitation+review of medication and eye exam” (31).

In the moderate risk table (16), a value between 5-3 is established, indicating the following procedures: “preventive physiotherapy+repeat the test in 3 months and! or revision of medication and! or eye exam” (32).

In the low risk table (17), a value between 2-1is established, indicating t he following procedures: “walk three times a week+application to control the walk+repeat the test in three months” (33).

In the risk-free table (18), the value zero is established, indicating the following procedures: “walk three times a week+repeat the test in six months” (34).

If the indicated prevention includes preventive physical rehabilitation, the same test can be performed in physiotherapy sessions, the result of processing the motor parameters for risk (Rosenstein method) and gait and balance (Fusion Madgwick followed by processing gait and balance indexes), that is, prediction of risk of falling (10) can be used to quantify the clinical practice and evolution of preventive treatment in the clinical environment (4), in real time, at a low cost, determining whether the risk of falling is moderate (16) or low (17), or even if there is an absence (18) of risk of falling. Likewise, if the clinician, in the screening, understands that the risk is very high (15), and in addition to preventive rehabilitation, should include the user's home monitoring, then this user downloads the application on their own cell (2) and of a responsible family member (F) and uses the system continuously (the sensor (1) attached to the body or the cell itself attached to a strap). When the risk is measured in continuous monitoring by algorithm, the apparatus vibrates and beeps and the patient (P) has the opportunity to act before the fall. The family member (F) can also download the same application and, using a specific password, monitor, even from work, the movement of the elderly who stayed at home, monitor the activity performed by the elderly at that moment (lying, sitting, walking) or become aware of a preventive fall or fall alarm for that family member.

Therefore, according to the present invention, the inventive solution is in the composition of solutions involving method and system, improved with the use of specific signal processing, leading to the clinical environment (4) and everyday environment (5), a low cost solution and easy to understand, measurable in graphs and numbers to the detriment of the current clinical approach based on subjective evaluation, or the current state of the art that contemplates fall prevention only from the motor point of view, and not systemic as recommended in scientific evidence.

1) SYSTEM FOR THE PREVENTION AND PREDICTION OF POSTURAL FALL RISK to prevent falls through an objective tool in the clinical environment (4) and everyday environment (5), based on clinical and motor information for the assessment, screening and monitoring of the risk of falling and motor performance with encouragement for the practice of preventive physical rehabilitation, offering systemic and interdisciplinary coverage to support clinical decision-making and remote monitoring of the user, to impact fall prevention, improve the quality of life of the elderly and intelligent management of health resources due to the decrease in the rate of hospitalizations and costs with falls in the hospital, clinical and domestic environment, characterized by the fact that the system includes patient (P) for collecting clinical data (D) and motor data (D1) with the dedicated application and sensor (1) (cell or Inertial sensor), according to which the therapist (T) collects information processed by a binary method that classifies as positive or negative, the prevention or absence of the risk assessed in a questionnaire, said therapist (T) asks the patient (P) to perform tasks including anticipatory and compensatory reactions, sensory guidance and dynamic gait balance; the patient (P) uses the sensor (1) coupled to the body, which automatically processes the inertial signal of the tasks to generate the risk indexes of falling, walking and balance, and classifying the level of risk as low, moderate or high, through processing (PI), and part of the algorithms is embedded in the sensor (1) of the cell (2) and the other part is processed in the cloud (3), instantly in the presence of the wireless signal, via Bluetooth connection, or as soon as possible, as the system enters wireless coverage; the patient (P) has their risk constantly controlled by continuous and automatic calculation of the risk, with partial processing autonomy (PI) between the apparatus and the patient's (P) cell (2); for prevention, the Rosenstein index rate reduction trigger triggers the vibration or beep of the apparatus to request the patient's motor attention (P); the user's motor behavior can also be monitored remotely, from cloud processing (3), by the therapist (T) or family member (F).

2) SYSTEM FOR PREVENTION AND PREDICTION OF POSTURAL FALL RISK, according to claim 1, characterized by the fact that it includes a clinical environment (4) and a daily environment (S), and part of the data (4) is offline (6) and in the cloud (3); among the related data are the clinical data (D) APP (7), the inertial data 9D (8), the inertial data 9D merged by the signal processing by quaternions (9), the inertial data 9D (8) communicating with Rosenstein prediction of fall risk (10) and the data merged by the signal processing by quaternions (9) with the gait and balance algorithms (11), with a communication of these with the gait and balance indexes (12) available in real time to support preventive assessment and rehabilitation; in the clinical and domestic environment (4) there is also the screening (13) of fallers by risk level with the suggestion of clinical guidance, this communicator with the risk monitoring (14) in the everyday environment (S) with preventive vibration or beep biofeedback in the continuous use of a wearable device.

3) SYSTEM FOR PREVENTION AND PREDICTION OF POSTURAL FALL RISK, according to claims 1 and 2, characterized by the fact that the results are achieved from the raw motion data from accelerometers (A1), gyroscopes (GI) and magnetometers (M!) of the inertial sensor (1), (cellular (2) or sensor (1) inertial) coupled to the patient's body (P) in the clinical assessment of the risk of falling, or in the same way, continuously coupled to the domestic patient (P).

4) METHOD of integrating clinical and motor information obtained in the system of claims 1 to 3, characterized by the integration of clinical data (D) APP (7) (clinical information) and gait and balance algorithms (11) (objective gait indexes and balance), by application and processing of measured vectors of the inertial signal, used on the patient's body (P) for clinical testing—prediction of the level of risk of falling (10) (Rosenstein risk—performed by a technician in the clinical environment (4) referring the patient (P) to the most appropriate treatment; these combined items generate the calculation of the risk of falling, generating a result (R1) that may indicate high risk (1S), moderate risk (16), low risk (17) or no risk (18).

5) METHOD, according to claim 4, characterized by the fact that in the table clinical data (D) APP (7) the patient (P) is subjected to a test (19) based on clinical data (D) APP (7), generating the evaluation (20) and the results: risk of falling+=3 (21), risk of falling+=2 (22), risk of falling+=1 (23) and risk of falling+=0 (24); then, in the gait and balance algorithms (11), the patient (P) is subjected to a test2 (2S) based on these indicators, generating the “motor parameters” (26) and the results: risk of falling+=3 (21) and risk of falling+=0 (24); in the table prediction of the level of risk of falling (10) (Rosenstein) the patient (P) is submitted to a test3 (27) that leads to the risk prediction (10B), with the results: risk of falling+=3 (23) and risk of falling+=0 (24).

6) METHOD, according to claim 4, characterized by the fact that after the assessment and calculation of the risk of falls, the result (R1) generated produces a risk stratification, in the table shown as high risk (15) a value >6 where there are two options: high risk 12-9 (28) and high risk 8-6 (29), both stratification ranges followed by appropriate clinical guidelines.

7) METHOD, according to claim 4, characterized by the fact that in the moderate risk frame (16) a value is set between 5-3 and in the low risk frame (17) a value is set between 2-1, also followed by adequate guidelines to support clinical decision.
